(19) United States
(12) Patent Application Publication
(10) Pub. No.: US 2018/0310862 A1
(43) Pub. Date: Nov. 1, 2018

(54) Device And Mechanism For Facilitating Non-Invasive, Non-Piercing Monitoring Of Blood Hemoglobin

(71) Applicant: Tech4Life Enterprises Canada, Inc., Oakville (CA)

(72) Inventors: Shariq Ramzan Ali Khoja, Oakville (CA); Muhammad Abdul Muqeet, Karachi (PK); Syeda Suha Tirmizi, Redmond, WA (US); Hafiz Imtiaz Ahmed, Karachi (PK); Muhammad Sabiq Eijaz Hussain, Karachi (PK)

(21) Appl. No.: 16/029,375

(22) Filed: Jul. 6, 2018

Related U.S. Application Data
(63) Continuation of application No. 14/424,947, filed on Feb. 27, 2015, now abandoned.
(60) Provisional application No. 61/946,576, filed on Feb. 28, 2014.
(60) Provisional application No. 61/946,580, filed on Feb. 28, 2014.

(51) Int. Cl. A61B 5/145; A61B 5/00; A61B 5/1455

(52) U.S. Cl. CPC A61B 5/14532; A61B 5/742; A61B 5/1495; A61B 5/6838; A61B 5/1455; A61B 5/7278

(57) ABSTRACT

A mechanism is described for facilitating non-invasive and non-skin piercing monitoring of blood hemoglobin according to one embodiment. A method of embodiments, as described herein, includes receiving a body part including a finger, where the body part in the placement area causes one or more sets of interruptions in the running of a light, where each set of interruptions includes a plurality of interruptions, and detecting initial readings corresponding to interruptions of the one or more sets of interruptions, the initial readings including signals, where an analog signal is generated each time the light is interrupted while passing through the body part. The method may further include calculating absolute values based on average raw values, where calculating includes computing an average absolute value based on the absolute values, and computing a final hemoglobin reading based on the average absolute value.

### PRIORITY

This application is a continuation of U.S. patent application Ser. No. 14/424,947, filed as a U.S. National Stage Application, under 35 U.S.C. § 371, of International Application No. PCT/US2014/041789, filed Jun. 10, 2014, which claims the benefit of and priority to U.S. Provisional Application No. 61/946,576, filed Feb. 28, 2014, and U.S. Provisional Application No. 61/946,580, filed Feb. 28, 2014, each of which applications is incorporated by reference in its entirety into this application.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

Embodiments described herein generally relate to computing devices. More particularly, embodiments relate to a device having a mechanism for facilitating non-invasive, non-piercing monitoring of blood hemoglobin

### BACKGROUND

Globally, anemia (also known as low hemoglobin or blood count) affects 1.62 billion people. The highest prevalence is in preschool-age children (293 million). Approximately 56 million pregnant women are affected by anemia. Anemia is known to cause 3.7% deaths in Africa, and about 12% deaths in Asia. Anemia can reduce a woman's ability to withstand the adverse effects of blood loss during and after Pregnancy. Obstetric hemorrhage is estimated to cause 25% of all maternal deaths and is the leading direct cause of maternal mortality worldwide. In Africa and Asia, where most maternal deaths occur, post-partum hemorrhage accounts for more than 30% of all maternal deaths.

Effective screening programs in prenatal and postnatal programs have been hampered by the lack of simple, safe, accurate, low-cost hemoglobin testing tools. The majority of women who suffer from anemia live in low-resource areas where accurate diagnostics are unavailable. In such settings, anemia often goes undetected and untreated. A noninvasive device could offer major advantages over current methods and approaches and would expand access to screening and increase early identification and treatment of anemia. The commonly used laboratory-based methods are expensive, complex, and necessitate a blood draw, requiring a trained technical person. Further, for many patients, this process can be painful and inconvenient and such invasive and painful processes are one of the reasons for poor patient compliance both with treatments and overall disease self-management.

### SUMMARY OF THE INVENTION

In accordance with embodiments, there are provided mechanisms and methods for facilitating non-invasive and non-skin piercing monitoring of blood hemoglobin according to one embodiment. In one embodiment and by way of example, a method includes receiving a body

part including a finger, where the body part in the placement area causes one or more sets of interruptions in the running of a light, where each set of interruptions includes a plurality of interruptions, and detecting initial readings corresponding to interruptions of the one or more sets of interruptions, the initial readings including signals, where an analog signal is generated each time the light is interrupted while passing through the body part. The method may further include calculating absolute values based on average raw values, where calculating includes computing an average absolute value based on the absolute values, and computing a final hemoglobin reading based on the average absolute value.

### BRIEF DESCRIPTION OF DRAWINGS

Embodiments are illustrated by way of example, and not by way of limitation, in the figures of the accompanying drawings in which like reference numerals refer to similar elements.

FIG. 1 illustrates a computing device (e.g., hemoglobin monitoring device) hosting a non-invasive hemoglobin monitoring mechanism and non-invasive hemoglobin monitoring elements according to one embodiment.

FIG. 2 illustrates a non-invasive hemoglobin monitoring mechanism and non-invasive hemoglobin monitoring elements according to one embodiment.

FIG. 3A illustrates a transaction sequence for facilitating non-invasive blood hemoglobin monitoring using a non-invasive hemoglobin monitoring device of FIG. 1 according to one embodiment.

FIG. 3B illustrates a transaction sequence for facilitating non-invasive blood hemoglobin monitoring using a non-invasive hemoglobin monitoring device of FIG. 1 according to one embodiment.

FIG. 3C illustrates a method for facilitating non-invasive blood hemoglobin monitoring using a non-invasive hemoglobin monitoring device of FIG. 1 according to one embodiment.

FIG. 4A illustrates a front/side view of a monitoring device of FIG. 1 according to one embodiment;

FIG. 4B illustrates a side view of a monitoring device of FIG. 1 according to one embodiment;

FIG. 4C illustrates a back/top view of a monitoring device of FIG. 1 according to one embodiment;

FIG. 4D illustrates an unassembled view of a monitoring device of FIG. 1 according to one embodiment;

FIG. 5 illustrates a computer system suitable for implementing embodiments of the present disclosure according to one embodiment.

### DETAILED DESCRIPTION

In the following description, numerous specific details are set forth. However, embodiments, as described herein, may be practiced without these specific details. In other instances, well-known circuits, structures and techniques have not been shown in details in order not to obscure the understanding of this description.

Embodiments provide for a low-cost, non-invasive, non-piercing anemia screening tool to bring positive, radical change to anemia screening and monitoring and support effective treatment practices as well as to make it easier to monitor hemoglobin count or iron levels of a person (e.g., healthy person doing a regular checkup, anemia patient, pregnant woman, etc.) to help reduce the risk of potentially life-threatening complications, such as during or shortly after childbirth, as well as preventing long-lasting health problems associated with severe anemia.

In low-resource health settings, it is found to be more complex to diagnose the most severe cases of anemia. Other, more quantitative point-of-care tests such as; require blood samples, maintenance of equipment, and recurrent supplies, limiting their use in resource-poor settings. Non-invasive Hemoglobin measurement, as provided by the embodiment, may have many advantages including, but not limited to, the prevention of pain and potential transmission of infectious diseases, a reduced need for trained personnel, short measurement time, and the absence of bio-hazardous waste. It offers a unique and innovative solution for accurate and quick hemoglobin measurements.

Embodiments provide for a hemoglobin monitoring device having a non-invasive technique for monitoring hemoglobin count or levels that can be used for self-monitoring by a user, such as a healthy person, a patient suffering from anemia, or other caregivers in the family. This can potentially benefit a large population, especially women and children in the low or middle income countries (LMIC). Embodiments provide for a hemoglobin monitoring device that is designed for all age groups and genders suffering from anemia. Further, the hemoglobin monitoring device is formed such that people with low literacy or poor understanding of technology may be able to use it with fluency and obtain its benefits. Additionally, in one embodiment, the non-invasive hemoglobin monitoring device enhances screenings and regular testing of hemoglobin, thus reducing the burden of disease.

In one embodiment, the hemoglobin monitoring device helps patients take control of their disease as well as facilitates physicians to view and analyze complete hemoglobin profile for the respective patients. Further, in one embodiment, the device aims to increase SMBG compliance rate that can ultimately reduce the disease burden globally. Embodiments provide for measuring hemoglobin levels by using, for example, a near infrared technique, using, for example, set parameters and mathematical algorithms which are capable of correcting the received values according to the gold standard values.

It is contemplated that anemia refers to a decrease in the number of red blood cells (RBCs) or lower than normal quantity of hemoglobin in the blood. Hemoglobin (also referred to as “haemoglobin” or simply abbreviated as “Hb” or “Hgb”) refers to the iron-containing oxygen-transport metalloprotein in the RBCs of most vertebrates (e.g., humans and other mammals, reptiles, amphibians, etc.) and some invertebrates (e.g., worms, insects, etc.) and is responsible for carrying oxygen from respiratory organs (e.g., lungs, gills, etc.) to the rest of the body (e.g., tissues, etc.) where it releases the oxygen to burn nutrients to provide energy to power the organism functions (e.g., process of metabolism).

It is to be noted that embodiments provide for a novel and innovative monitoring of blood hemoglobin that is non-invasive, such as without having to pierce or pinch or poke a skin (e.g., human skin, animal skin, etc.) for drawing blood for blood hemoglobin testing or monitoring purposes. Conventional devices require that the skin (e.g., finger) be pierced (or pinched or pricked or poked) with a sharp needle-like instrument to draw one or more drops of blood that can then be used for performing necessary tests to determine the blood hemoglobin level. Other conventional techniques involve using intimidating and invasive laboratory devices.

It is to be further noted that embodiments are not limited to merely hemoglobin monitoring and that any number and type of monitoring (also referred to as “detecting”, “observing”, or “testing”), may be performed, such as monitoring of glucose, heart rate, blood pressure, body temperature (e.g., fever), etc. Furthermore, embodiments are not merely limited to humans and that the aforementioned monitoring (e.g., monitoring of hemoglobin, glucose, heart rate, blood pressure, body temperature (e.g., fever), etc.) may be performed on any number and type of animals.

Throughout this document, terms like “logic”, “component”, “module”, “framework”, “engine”, “mechanism”, “technique”, “element”, and/or the like, may be referenced interchangeably and include, by way of example, software, hardware, and/or any combination of software and hardware, such as firmware. Further, any use of a particular brand, word, term, phrase, name, acronym, or the like, such as “hemoglobin meter”, “self-monitoring of blood hemoglobin” or “SMBG”, “lower or middle income countries” or “LMIC”, “blood hemoglobin monitoring device”, and/or the like, should not be read to limit embodiments to software or devices that carry that label in products or in literature external to this document. Further, for the sake of brevity, clarity, and ease of understanding, certain devices, techniques, methods, materials, conditions, diseases, etc., may be referenced by name or their acronym while other are ignored; however, it is to be noted that embodiments are not limited to these or any other particular devices, techniques, methods, materials, conditions, diseases, etc., and that embodiments are applicable and compatible to and workable with all forms, manners, brands, types and numbers of devices, techniques, methods, materials, conditions, diseases, etc.

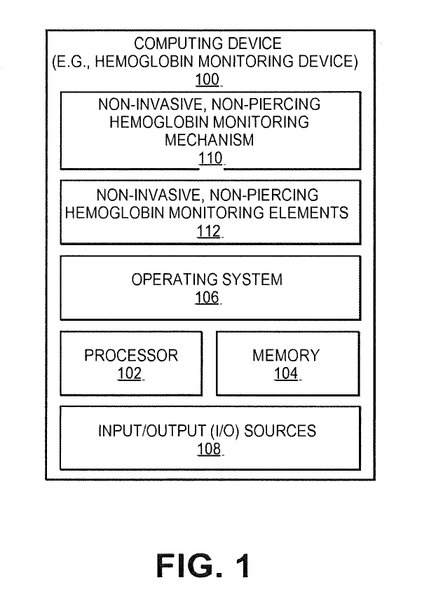

FIG. 1 illustrates a computing device 100 (e.g., hemoglobin monitoring device) hosting a non-invasive hemoglobin monitoring mechanism 110 and non-invasive hemoglobin monitoring elements 112 according to one embodiment. Computing device 100 serves as a host machine for employing non-invasive hemoglobin monitoring mechanism (“monitoring mechanism”) 110 and non-invasive hemoglobin monitoring elements (“monitoring elements”) 112 for non-invasive blood hemoglobin monitoring, including self-monitoring of hemoglobin level. Throughout the document, computing device 100 may be interchangeably referred to as (but not limited to) “host machine”, “hemoglobin meter”, “hemoglobin device”, “hemoglobin monitoring device”, “non-invasive blood hemoglobin monitor” “non-invasive blood hemoglobin monitor”, “hemoglobin monitor”, “monitoring device”, simply “device” or “monitor”.

It is contemplated that blood hemoglobin monitoring refers to testing of concentration of hemoglobin in the blood and it is particularly important for patients with anemia. Embodiments provide for monitoring device 100 having monitoring mechanism 110 and monitoring elements 112 for facilitating non-invasive/non-piercing monitoring of blood hemoglobin, where the non-invasive monitoring is performed without having to pierce or prick the skin (e.g., finger) or having the need for drawing blood.

Although throughout this document monitoring device 100, monitoring mechanism 110, and monitoring elements 112 are discussed with reference to hemoglobin monitoring in humans, it is contemplated and in some embodiments, monitoring device 100, monitoring mechanism 110, and monitoring elements 112 are not limited to monitoring of hemoglobin or humans and that they may be used for monitoring of other conditions in humans, such as hemoglobin, heart rate, blood pressure, body temperature (e.g., fever), etc., and similarly, in some embodiments, monitoring device 100, monitoring mechanism 110, and monitoring elements 112 are not limited to merely humans and that they may be used for monitoring of various conditions, such as hemoglobin, glucose, heart rate, blood pressure, body temperature (e.g., fever), etc., in animals.

Computing device 100 may include large computing systems, such as server computers, desktop computers, etc., and may further include set-top boxes (e.g., Internet-based cable television set-top boxes, etc.), global positioning system (GPS)-based devices, etc. Computing device 100 may include mobile computing devices, such as cellular phones including smartphones (e.g., iPhone® by Apple®, BlackBerry® by Research in Motion®, etc.), personal digital assistants (PDAs), tablet computers (e.g., iPad® by Apple®, Galaxy 3® by Samsung®, etc.), laptop computers (e.g., notebook, netbook, Ultrabook™ system, etc.), e-readers (e.g., Kindle® by Amazon®, Nook® by Barnes and Nobles®, etc.), etc.

Computing system 100 may serve as a hemoglobin monitoring device employing or hosting monitoring mechanism 110 which may be accessed by a user directly (such as by placing a finger in a dedicated finger placement area) or through one or more other computing devices (such as mobile computing devices, such as a smartphone, a tablet computer, a laptop computer, etc.). The term “user” may refer to an individual or a group of individuals (e.g., end-users, such as human beings including, but not limited to, patients, doctors, nurses, laboratory technicians, etc., administrative users, such as software programmers, system administrators, laboratory or office managers, etc.) who can access (to use or alter) various features of monitoring mechanism 110. Monitoring mechanism 110 may be offered as a software program or application (e.g., a downloaded or cloud-based application, such as a business application, a website, etc.) at computing device 100 or one or more of the other computing devices accessible to the user.

Computing device 100 includes an operating system (OS) 106 serving as an interface between any hardware or physical resources of the computer device 100 and a user. Computing device 100 further includes one or more processors 102, memory devices 104, network devices, drivers, or the like, as well as input/output (I/O) sources 108, such as touchscreens, touch panels, touch pads, virtual or regular keyboards, virtual or regular mice, etc. It is to be noted that terms like “node”, “computing node”, “server”, “server device”, “cloud computer”, “cloud server”, “cloud server computer”, “machine”, “host machine”, “device”, “computing device”, “computer”, “computing system”, and the like, may be used interchangeably throughout this document. It is to be further noted that terms like “application”, “software application”, “program”, “software program”, “package”, and “software package” may be used interchangeably throughout this document. Similarly, terms like “job”, “input”, “request” and “message” may be used interchangeably throughout this document.

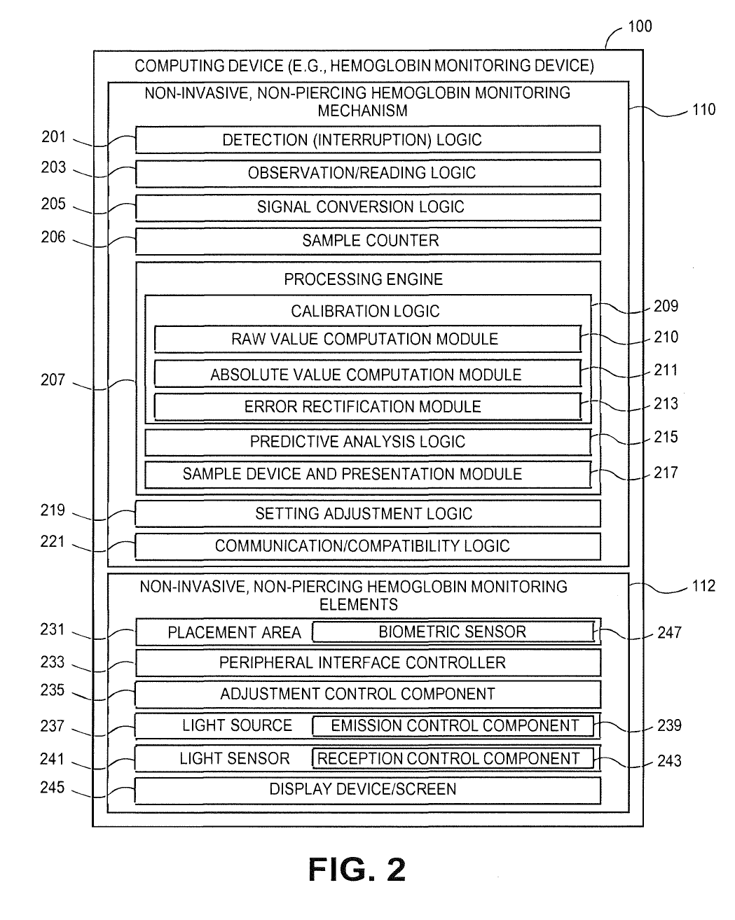

FIG. 2 illustrates a non-invasive hemoglobin monitoring mechanism 110 and non-invasive hemoglobin monitoring elements 112 according to one embodiment. In one embodiment, monitoring mechanism 110 includes any number and type of components, such as (1) detection (interruption) logic 201; (2) observation/reading logic 203; (3) signal conversion logic 205; (4) sample counter 206; (5) processing engine 207 including (a) calibration logic 209 having (i) raw value computation module 210, (ii) absolute value computation module 211, and (ii) error rectification module 213, (b) predictive analysis logic 215, and (c) sampling device and presentation logic (“presentation logic”) 217; (6) settings adjustment logic (“adjustment logic”) 219; and (7) communication/compatibility logic 221. In one embodiment, monitoring elements 112 include placement area 231 having biometric sensor 247; peripheral interface controller (“interface controller”) 233; adjustment control component (“adjustment component”) 235; light source 237 including emission control component (“emission component”) 239; sensor 241 including reception control component (“reception component”) 243; and display screen 245.

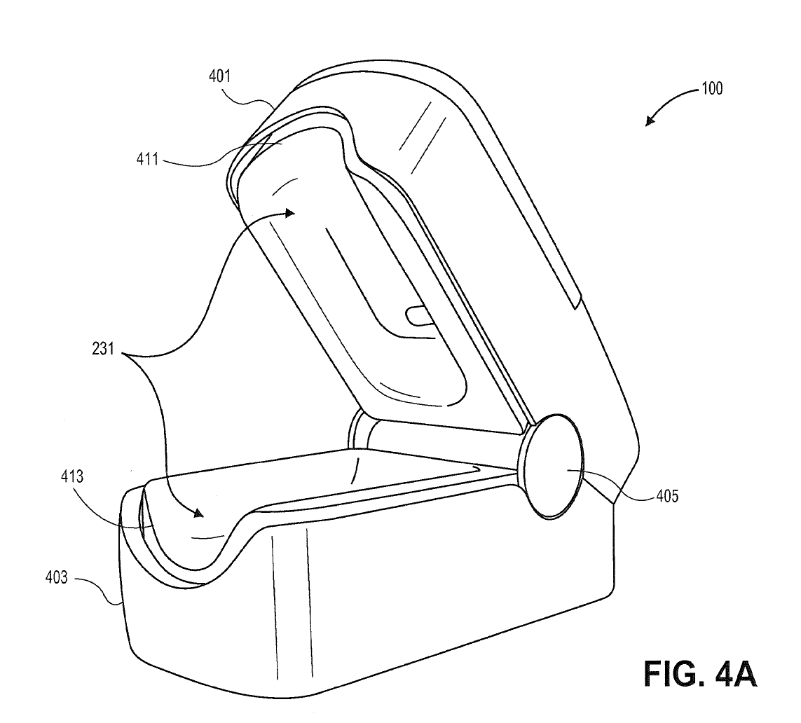

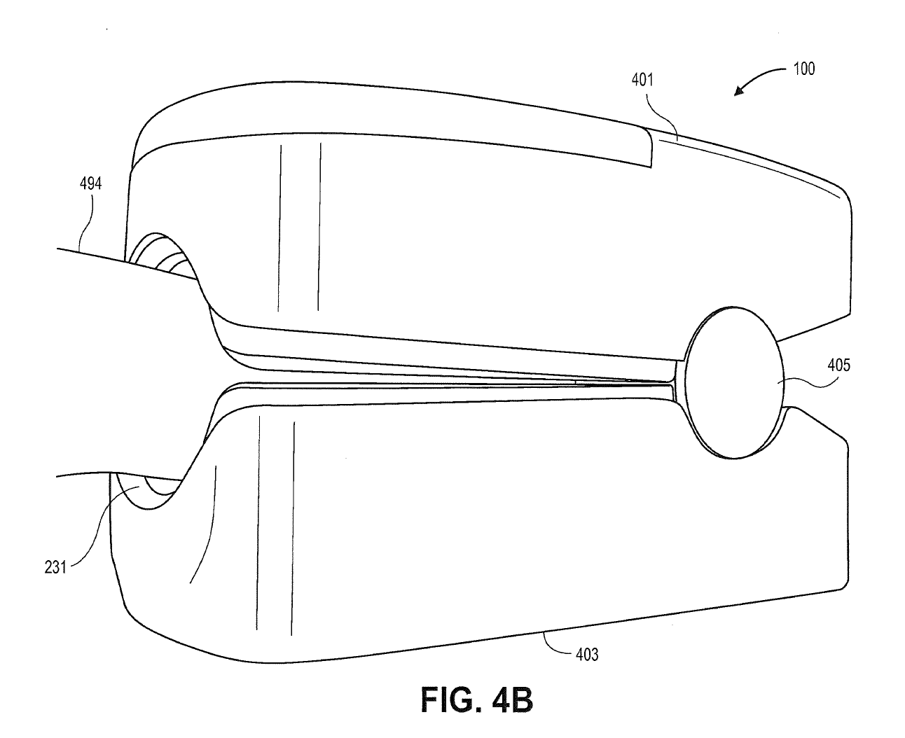

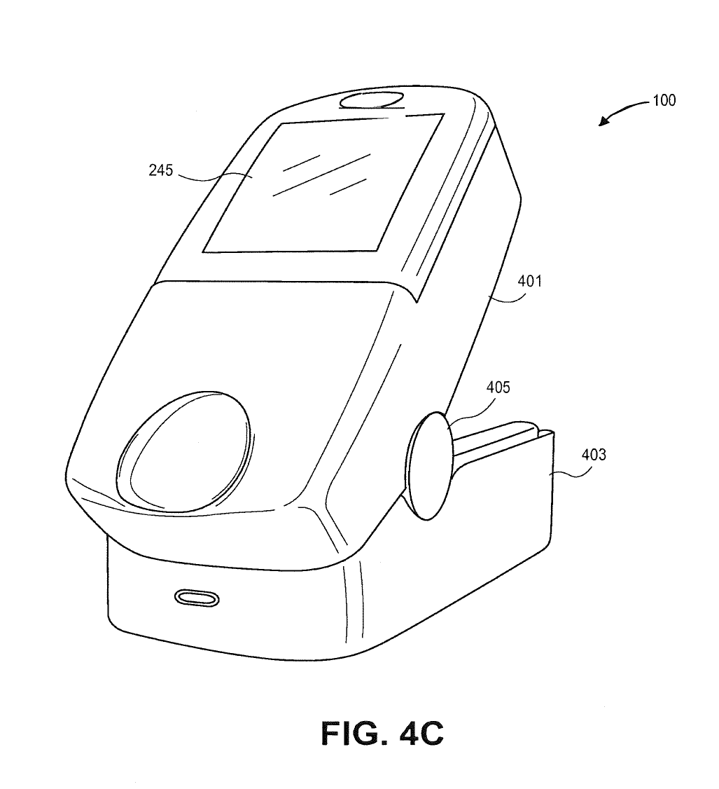

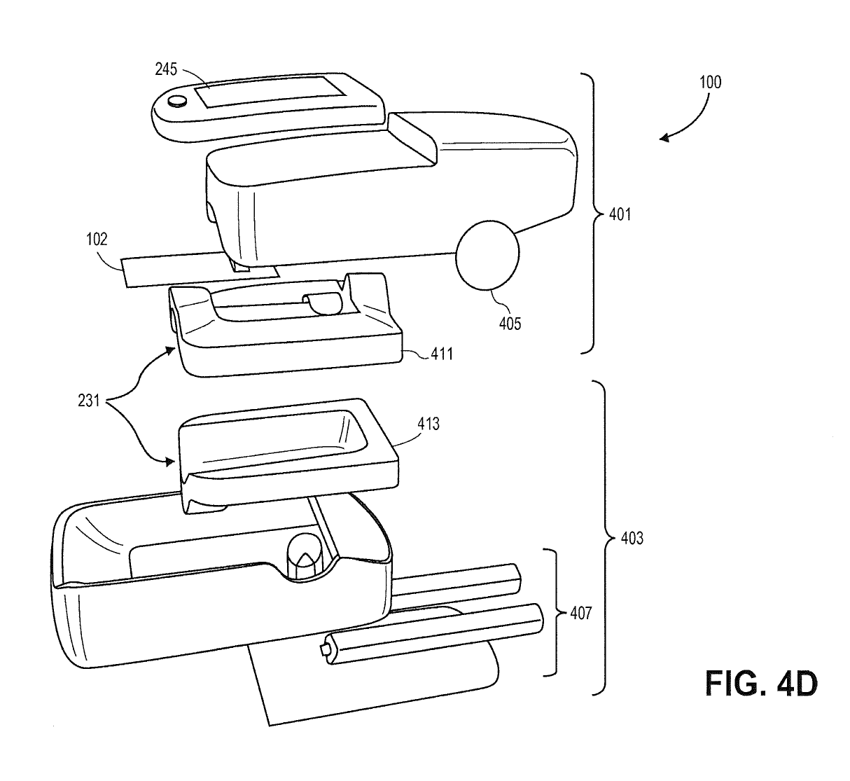

Embodiments provide for monitoring device 100 having monitoring mechanism 110 and monitoring elements 112 for facilitating non-invasive/non-piercing monitoring of blood hemoglobin, where the non-invasive monitoring is performed without having to use intimidating laboratory equipment or to pierce or prick the skin (e.g., finger) or having the need for drawing blood. In one embodiment, upon turning on monitoring device 100, such as by turning on an on/off switch, a light begins to emit from emission component 239 of light source 237 which may be placed within a top or upper chamber (also referred to as “lid”, “portion”, “half or “section”), such as top chamber 401 of FIG. 4A, of device 100. The emitted light may then travel down to a bottom or lower chamber (similarly, also referred to as “lid”, “portion”, “half or “section”), such as bottom chamber 403 of FIG. 4B, of device 100 where the light may be received by reception component 243 of photo or light sensor 241 that is placed in the bottom chamber of device 100.

In one embodiment, the light may include an infrared light emitted from emission component 239 (e.g., an infrared light-emitting diode (“LED”), such as a two-lead semiconductor, etc.) of light source 237 and received at reception component 243 (e.g., phototransistor or light receptor, such as L14G1/2/3, a silicone phototransistor hermetically sealed package with a combination of, for example, LED55B/55C or LED56 Gallium Arsenide (GaAs)) of light sensor 241, where the light may be transmitted over a wavelength and is passed through a couple of beams, such as an emission beam (as facilitated by emission component 239) and a reception beam (as facilitated by reception component 243).

Further, the light may be transmitted over a peak emission wavelength (such as 940 nm, etc.) and an emission angle, such as +8 degrees at ½ power, etc. It is contemplated that embodiments are not limited to any particular wavelength (e.g., a peak wavelength may be chosen from any range of wavelengths, such as from 640 nm to 1000 nm, etc.) but for the sake of brevity and ease of understanding, throughout this document, a particular wavelength, such as 940 nm, may be regarded as associated with the light and referred to as the peak emission wavelength. Further, in some embodiments, wavelengths may be adjusted within device 100 supporting one or more wavelengths as deemed necessary and appropriate for performing fine hemoglobin monitoring and subsequently, producing accurate hemoglobin readings.

Once monitoring device 100 has been turned on and the light has started to travel, a person may place their finger (or thumb, toe, etc.) into placement area 231 which, as a result, may then interrupt the light running between light source 237 and sensor 239. In one embodiment, this interruption and the light passing through the finger may be detected by detection (interruption) logic 201 and received at interface controller 233 (e.g., 8 bit peripheral interface controller, such as Atmega328/328P, etc.) in the form of an analog signal and further, this interruption generates an observation or reading which may be detected or read by observation/reading logic 203. It is contemplated that at this level of monitoring, the observations (also referred to as “observation readings” or “initial readings”) may be made in an analog signal form and continue for a given time period and/or a number of observations set forth at device 100. For example and in one embodiment, monitoring device 100 may be set to facilitate observation/reading logic 203 to obtain a fixed number or range of observation readings over an unlimited period of time (such as set to obtain 5 observations, 10 observations, 40-50 observations, etc.) or an unlimited number of observation readings over a fixed period or range of time (such as set to obtain any number of values for 5 seconds, 10 seconds, 30-60 seconds, etc.).

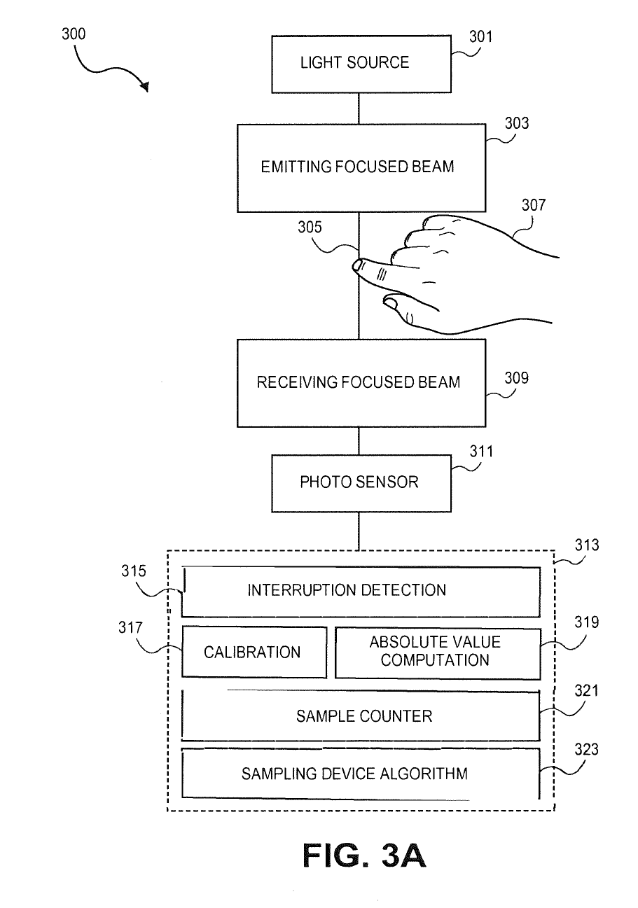

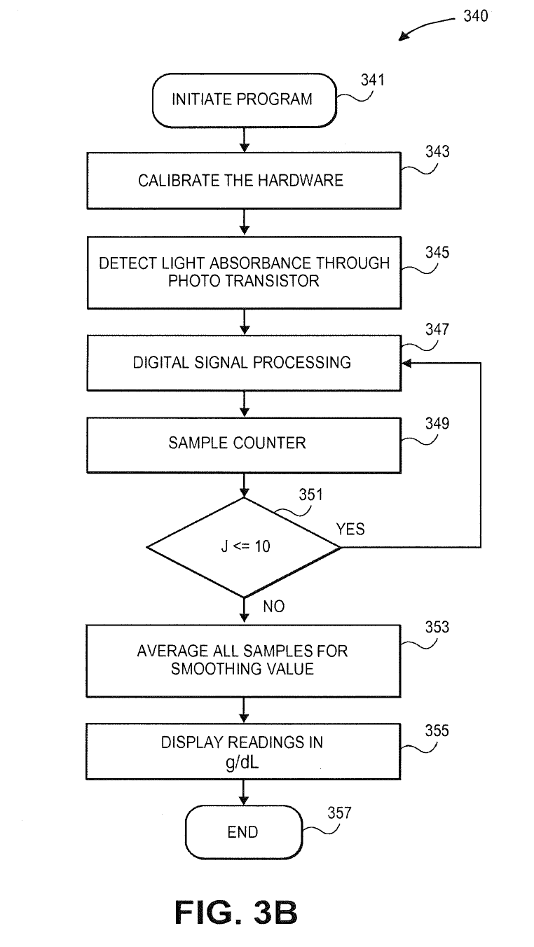

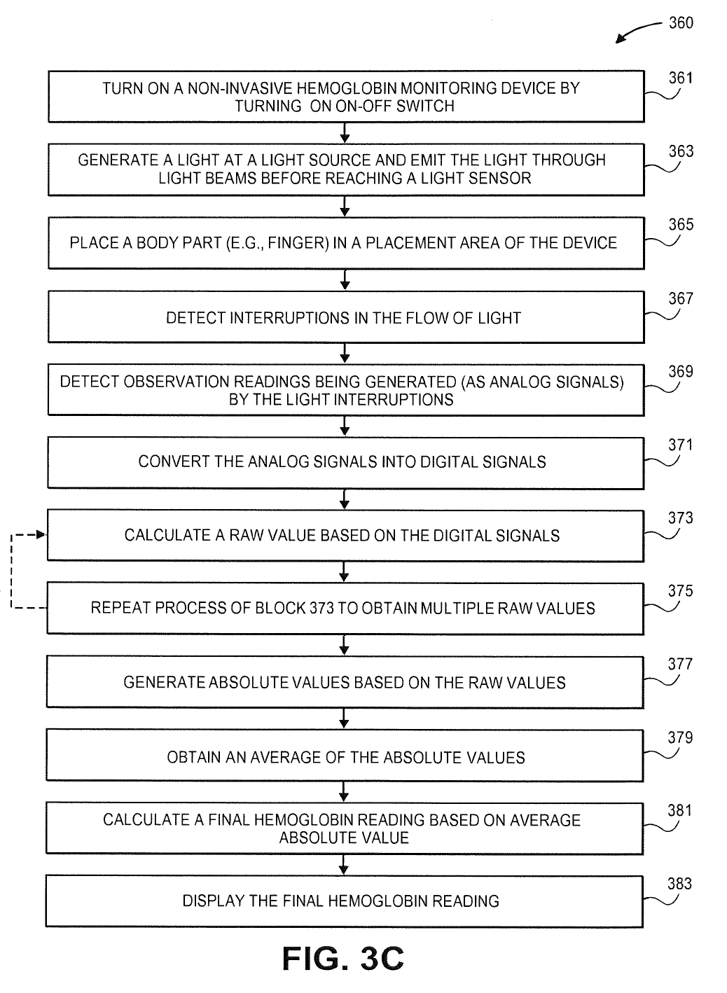

In one embodiment, sample counter 206 may be used to set a count of the number of interruptions that are to be used for deriving observation readings and subsequently, determining the final hemoglobin reading of hemoglobin concentration in the blood. For example, monitoring device 100 may be set or modified, such as via setting adjustment logic 219, to facilitate or take a certain number of interruptions that may be grouped into a number interruption sets, such as 5 sets of interruptions may be taken for processing which each set of interruptions may include 10 interruptions for a total of 50 interruptions grouped into 5 sets of interruptions. It is contemplated that each predefined to have the same or a different number of interruptions, such as in an ascending order or a descending order or without any particular order. Accordingly, in one embodiment as described with reference to FIG. 3B, sample counter 206 may be assigned a particular value, such as 10, representing, for example, a number of interruptions (or sets of interruptions) that are to be taken such that sample counter 206 allows the process to continue to iterate or repeat until the value is reached and then sample counter 206 stops the process. The process may then be restarted for another set of interruptions and/or when another user/patient and accordingly, sample counter 206 may be reset to begin again.

It is contemplated that embodiments are not limited to any of the wavelengths, emission angels, observation readings, time periods, etc., and that any of such values may be set to be fixed, varied, or adjusted or modified, etc., as deemed necessary or appropriate by or based on, for example, updated research, medical opinions, medical personnel, patient or end-user needs, etc. In one embodiment, these settings, variances, and adjustments, etc., may be programmed-in as default values and/or set at the time of manufacturing while, in another embodiment, these values may be set or changed via settings adjustment logic 219 as facilitated by an external or physical adjustment component 235 that is capable of being used by an individual (e.g., system administrator, computer programmer, medical personnel (such as a doctor, a nurse, etc.), etc.).

Placing the finger at placement area 231 and into the light path going through the insulated emission and receiving cavities may reduce the effects of the external light causing variation in the wavelength at the time of the light being received at reception component 243. In some embodiments, placement area 231 may include an optional biometric sensor 247 to sense certain biometric features of the person, such as fingerprints, medical history, glucose level, etc., to have and maintain hemoglobin reading data relating to each person using device 100. This variation may be considered an interruption and used for calibration that may set the absolute value for the next coming signals for the same finger. This signal may then be converted into digital information in the form of numbers that may be treated by mathematical algorithms using predictive values of the sample.

For example and in one embodiment, as aforementioned, any number of interruptions are sensed in a wave form (e.g., analog signal) and noted as observation readings by observation/reading logic 203. These readings are then provided to signal conversion logic 205 where the analog signals are converted into digital signals for further evaluation and processing by processing engine 207. For example, if device 100 was preset such that observation/reading logic 203 was programmed to take 5 observation readings on the same finger over a time period of 5 seconds, then these 5 observation readings may all be converted from their corresponding analog signals to digital signals by signal conversion logic 205 before they are sent to processing engine 207.

At processing engine 207, in one embodiment, the digital signals are put through a calibration process including producing a number of raw values using raw value computation module 210. For example, using mathematical formulae and/or algorithm, a number of raw values may be extracted from the digital signals and then, an average raw value is obtained by averaging out the raw values using computing module 210. For example, 5 raw values may be extracted from the 5 digital signals and the 5 raw values may then be averaged to obtain a single average raw value. In one embodiment, the process of obtaining observation readings (e.g., 5 observation readings in 5 seconds) may be repeated a number of times and put the through the aforementioned processes to receive a number of average raw values. Continuing with the above example, the process of obtaining 5 observation values in 5 seconds may be repeated a number of times, such as 3 times, and accordingly, for example, the 3 sets of 5 analog signals obtained from 3 sets of 5 observation values may be converted into 3 sets of digital values which are then converted into 3 sets of 5 raw values and then into 3 average raw values based on the 3 sets.

In one embodiment, as part of the calibration process, the number of average raw values (e.g., 3 average raw values) may then be put through another set of processing (such as using mathematical formulae and/or algorithm) to obtain corresponding absolute values (e.g., 3 absolute values) using absolute value computation module 211. The calibration process may further include using error rectification module 213 for identifying and rectifying any errors encountered during calculation of absolute values by applying or introducing various coefficients to the calculation process so that proper absolute values may be produced. In one embodiment, as will be further described below, various components and algorithms (e.g., software programs, mathematical formulae, etc.) may be used to perform the various tasks of calibration logic 209 and other components 215, 217 of processing engine 207. In one embodiment, absolute value computation module 211 may be used to calculate an average of the absolute values. Using the above example, an average absolute value may be obtained for each of the 3 sets of 5 observation readings from which 3 average raw values are extracted and then 3 absolute values are obtained which are then divided by the number of absolute values (e.g., 3 in this example) to obtain the average absolute value.

In one embodiment, predictive analysis logic 215 performs additional processing to predictively compute the average absolute value into a final hemoglobin reading (also referred to as “final reading”). As will be further described below, in one embodiment, various components and algorithms (e.g., software programs, mathematical formulae, etc.) may be used to perform the various tasks of predictive analysis logic 215 to predictively obtain the final hemoglobin reading that represents or indicates the amount or content or concentration or level of hemoglobin in the blood. At sampling device and presentation logic 217, the final hemoglobin reading may then be prepared for presentation by at display screen 245. For example, the final reading may be presented in any number of presentation forms, such as purely in numbers, words/text or characters, colors (e.g., red for high or low, blue or green for normal, etc.), symbols (such as an up-down arrow showing a trend or a number higher or lower than a threshold number for a particular user (e.g., patient)), graphical presentations (e.g., line graph, pie chart, bar chart, etc.), etc.

Hemoglobin concentration in blood is typically measured gram/liter (g/L), gram/deciliter (g/dL), or mole/liter (mol/L), etc. For example, hemoglobin concentration range for a normal man may be 13.8 to 18.0 g/dL, a normal woman may be 12.1 to 15.1 g/dL, a normal child may be 11 to 16 g/dL, a pregnant woman may be 11 to 14 g/dL, etc. In some embodiments, a final hemoglobin reading may be displayed on display screen 245 on its own or along with any number or type of other sets of data, such as a green circle or a smiley face for normal reading, a written or textual statement, such as “normal level”, etc., person's name, final reading history, etc. Similarly, an abnormal hemoglobin level reading for an anemic person may be displayed on display screen 245 on its own or along with any number and type of other sets of data, such as a red flashing light for warning, a written or textual statement, such as “anemic condition detected”, etc., person's name, final hemoglobin reading history, etc.

In one embodiment, the final hemoglobin reading may be displayed on display screen/device 245 (as shown in FIG. 4C) and it is contemplated that embodiment are not limited to any particular type of display screen 245 may include any number and type of display screens or devices, such as (but not limited to) liquid crystal display (CLD) display, organic light-emitting diode (OLED) display, light-emitting diode (LED) display, electroluminescent display (ELD), plasma display panel (PDP), surface-conduction electron-emitter display (SED), carbon nanotubes, quantum dot display, interferometric modulator display (IMOD), etc.

### Additional Technical Description

In one embodiment, following techniques and/or algorithms may be employed to facilitate mechanism 110 and elements 112 to perform various tasks and functions as described above; however, it is contemplated that embodiments are not limited to merely the following techniques or algorithms.

### Acquisition Method

In some embodiments, the various techniques, components, and/or algorithms employed and used in the aforementioned processing of acquiring observation readings and absolute values and producing the final hemoglobin readings may use (but not limited to) one or more of the following:

### Lambert's Law

The proportion of incident light absorbed by a transparent medium may be independent of the intensity of the light (such as provided that there is no other physical or chemical change to the medium) and accordingly, successive layers of equal thickness may transmit an equal proportion of the incident energy.

### Beer's Law

The absorption of light may be directly proportional to both the concentration of the absorbing medium and the thickness of the medium in the light path. A combination of the two laws (e.g., known jointly as the Beer-Lambert Law) may define the relationship between absorbance (A) and transmittance (T). In one embodiment, the light at the resonance wavelength of initial intensity, I₀, may be focused on the flame cell containing ground state atoms. The initial light intensity may be decreased by an amount determined by the atom concentration in the flame cell and the light may then be directed onto the detector where the reduced intensity, I, is measured. In one embodiment, the amount of light absorbed may be determined by comparing I to I₀.

Further, several related terms may be used to refer to the amount of light absorption that may have taken place. For example, “transmittance” may be used to refer to the ratio of the final intensity to the initial intensity and serve as an indication of the fraction of the initial light which passes through the flame cell to fall on the detector. Similarly, “percent transmission” may refer to the transmittance expressed in percentage terms, such as:

$$\% T = 100 \times \frac{I}{{I}_{0}}$$

These terms are easy to visualize on a physical basis, such as “absorbance” may refer to a mathematical quantity, such as:

$$A = log ( \frac{{I}_{0}}{I} )$$

Further, absorbance may refer to characterizing light absorption in absorption spectrophotometry, as this quantity may follow a linear relationship with concentration. Beer's Law may be used to define this relationship as:

A=abc

In A=abc, A may refer to the absorbance, where a may refer to the absorption coefficient, a constant which is characteristic of the absorbing species at a specific wavelength, where b may refer to the length of the light path intercepted by the absorption species in the absorption cell, and where c may refer to the concentration of the absorbing species. Further, this equation states that the absorbance may be directly proportional to the concentration of the absorbing species for a given set of instrumental conditions.

### Source Handling

In on embodiment, near Infra-Red (Near-IR) spectroscopy may be used by mechanism 110 and/or elements 112 to perform non-invasive blood hemoglobin monitoring. For example, NIR diffuse reflectance spectroscopy may involve the illumination of a spot on the body with low-energy near-IR light (e.g., 750-2500 nm), where the light may be partially absorbed and scattered, according to its interaction with chemical components within the tissue of the finger, before being transmitted to be detected by detection (interruption) logic 201.

### Infrared Spectroscopy

In one embodiment, spectroscopy may be used for identifying molecules as each molecule may have its own characteristic band where radiation may be absorbed at a specific wavelength. In this case, for example, the hemoglobin absorption curves may be small and have artifacts from various layers of tissues. In one embodiment, mechanism 110 may perform one or more processes for monitoring hemoglobin using several absorption frequencies, where the light is partially absorbed and scattered, according to its interaction with chemical components within the tissue of the finger, before being reflected back to detection (interruption) logic 201. It is contemplated that detection (interruption) logic 201 may facilitate a detector (not shown) for detection of interruption, where the detector may be part of monitoring elements 112, such as independently placed or being part of light sensor 241.

### Light Scattering

In one embodiment, the skin of the finger may be radiated with infrared radiation and its scattering may be observed via observation/reading logic 203, wherein the presence of hemoglobin may change the effects of the scattering and thus providing a useful way of monitoring concentrations.

### Near-IR and Tissue Optical Properties

Furthermore, water, which is regarded as a major component of biological tissues, may have a simple infrared (IR) spectrum and a rich combination and overtone spectrum that can be extended into the near-IR. The assignment of the near-IR absorption bands for water may be used, where the intensity of the near-IR absorption bands for water may be sensitive to solute concentration and temperature. For example, it decreases as solute concentration increases because of the change in the molar ratio of water. The 600-1100 nm region of the spectrum may represent a window between the hemoglobin or glucose and visible absorption bands and water IR absorption, where the light can penetrate deep enough into the tissue to allow a spectral measurement or a therapeutic procedure. This spectral region may then be used for oxygen saturation, pulse oximetry, laser-Doppler flow measurements, etc.

Furthermore, processes, such as transport equation and diffusion theory, etc., may unfold description of the path of photons through human tissue as it expresses light propagation in tissues by a set of spectroscopic properties; the absorption coefficient, μα, the scattering coefficient, μs, the refractive index of the cells and the interstitial fluid; and the anisotropy factor g (the average cosine of the angle at which a photon is scattered). Another set of properties may include transport properties, such as the reduced scattering coefficient μs¹ where μs¹=μs[1−g]. The absorption coefficient, μα, equals the absorbance per unit path length, 2.303 εC cm-¹, where ε is the molar absorptivity and C is the molar concentration. The scattering coefficient μs=σρ where μα is the scattering cross-section and p is the number density of the particle. It has the same unit as μα (cm⁻¹) and is equivalent to the product of an absorptivity caused by scattering and the concentration of the scattering centers.

In one embodiment, various methods that are used for measuring the optical properties of tissues (e.g., μs, μα, and g) may include transmission, diffuse and localized reflectance, frequency domain measurement, etc.

### Absorption Characteristics of Hemoglobin in Tissues

The tissue combinations which absorb light in the spectral region of interest are known as chromophores. Each chromophore has its own individual absorption spectrum which describes the level of absorption at each wavelength. In the near-IR, known as the absorption window or therapeutic window, the major absorbing components in the soft tissues may include water, oxyhaemoglobin, deoxyhaemoglobin, etc., with minor contributions from other tissue chromophores, such as melanin, lipids, etc.

The concentration of water and melanin remains virtually persistent with time (e.g., static absorbers). On the other hand, the concentrations of dynamic absorbers, such as oxygenated and deoxygenated hemoglobin (such as related with blood oxygenation), and cytochrome oxidase (such as an enzyme in the oxidative metabolic pathway that provides an indicator of tissue oxygenation and cell metabolism), provide clinically useful physiological information. However, the concentration of cytochrome oxidase in tissue may be regarded as inferior when compared with hemoglobin (at least one order of magnitude below that of hemoglobin).

It is contemplated that hemoglobin may affect the measured transmitted or reflected signal by absorption of light at the overtone and combination band wavelengths, where light absorption may be expressed as I=Iₒe^{-μαl}, where l is the effective path length in the medium, and μα is the absorption coefficient. Further, changes in hemoglobin concentration may influence the measured μα of tissue through changes in absorption corresponding to water displacement (e.g., absorption decreases as hemoglobin concentration increases) or changes in its intrinsic absorption (e.g., absorption increases as hemoglobin concentration increases). Changes in μα because of water displacement may be nonspecific, and analytics with higher molecular weights may displace more water than is done by hemoglobin. Changes in the temperature and hydration status of the body may affect water absorption bands and act as noise sources for an NI hemoglobin measurement. The hemoglobin μα in the near-IR may be low and can be much smaller than that of water. However, its magnitude may be too small to allow for direct absorption measurements at wavelengths <1400 nm. Attenuation of light (<1400 nm) in a small body part, such as an average-sized human finger, may vary in the range of 3-4 absorbance units, and the expected change in absorbance because of a 5 mmol/L change in hemoglobin concentration may be ˜10-5 absorbance units.

### Effect of Hemoglobin on Tissue Scattering

Near-infrared light in the medical spectral window (˜700-900 nm) is weakly absorbed in tissue and is highly scattered similarly to the way that light is highly scattered in, for example, fog or in a glass of milk. This scattering is regarded as being at the core of near-infrared spectroscopy of tissue. The scattering properties of a sample are mainly determined by the size of the scattering particles relative to the wavelength of light and by the refractive index mismatch between the scattering particles and the surrounding medium. In biological tissues, the scattering centers include cells and cellular organelles. In the medical spectral window (700-1000 nm), cellular organelles have dimensions comparable to the wavelength, and their index of refraction is relatively close to that of the cytosol and extracellular fluid. As a result, light scattering in tissue is mainly forwarded directly and may show a relatively weak wavelength dependence. In strongly scattering media, the scattering properties may be described by the reduced scattering coefficient (μs′).

Changes in hemoglobin concentration may affect the intensity of light scattered by tissue, where the reduced scattering coefficient of a tissue can be expressed in a function form as:

$$μ {s}^{1} = f ( {p}_{1} {a}_{1} \frac{n cells}{n medium} )$$

Where p is the number density of scattering cells in the observation volume, a is the diameter of the cells, n cells is their refractive index, and n medium is the refractive index of interstitial fluid. Changes in the n medium may not be specific for a particular analysis and affected by any change in the total concentration of solutes in blood and interstitial fluid. During the hyperglycemic phase, the hemoglobin concentration may change frequently, whereas other analytic concentrations may change comparatively at a slower rate. It may be possible to relate δμs′ to changes in hemoglobin concentration over a short time span. The measured n^{th} water may decrease as the temperature increases. This can affect n cells/n medium in tissue and presents a source of error in scattering measurements. Values of μs′ are reported to decrease with the increasing concentrations of hemoglobin and other substances, such as sugar/glucose, in tissue-simulating phantoms because of their effect on n medium. Short Wavelength near infrared (640-1000 nm) spectra of aqueous solution of D-hemoglobin may be monitored, where the Observation yields that maximum absorption may occur in the range of 920-950 nm, so the selected wavelength for device 100 of FIG. 1 may be 940 nm and then used for non-invasive hemoglobin monitoring.

Monitoring device 100 may further include any number and type of touch/image components, where these touch/image components may include (but not limited to) image capturing devices (e.g., one or more cameras, etc.) and image sensing devices, such as (but not limited to) context-aware sensors (e.g., temperature sensors, feature measurement sensors, etc.) working with one or more cameras, environment sensors (such as to sense background colors, lights, etc.), biometric sensors, such as biometric sensor 247 (to detect fingerprints, etc.), and the like. Monitoring device 100 may also include one or more software applications to allow for sharing of user hemoglobin information with the user (e.g., patient), user's family members or friends, medical personnel (e.g., user's doctor, nurse, etc.), etc., via email, text, voice, social network web sites (e.g., Facebook®, Google+®, Twitter®, etc.), communication applications (e.g., Skype®, Tango®, Viber®, etc.), etc., offering one or more user interfaces (e.g., web user interface (WUI), graphical user interface (GUI), touchscreen, etc.) via display screen or device 245, while ensuring compatibility with changing technologies, parameters, protocols, standards, etc.

Communication/compatibility logic 221 may be used to facilitate dynamic communication and compatibility between monitoring device 100 and any number and type of other similar monitoring devices or other types of computing devices (such as a mobile computing device, a desktop computer, a server computing device, etc.), medical devices, storage devices, databases and/or data sources (such as data storage devices, hard drives, solid-state drives, hard disks, memory cards or devices, memory circuits, etc.), networks (e.g., cloud network, the Internet, intranet, cellular network, proximity networks, such as Bluetooth, Bluetooth low energy (BLE), Bluetooth Smart, Wi-Fi proximity, Radio Frequency Identification (RFID), Near Field Communication (NFC), Body Area Network (BAN), etc.), wireless or wired communications and relevant protocols (e.g., Wi-Fi®, WiMAX, Ethernet, etc.), connectivity and location management techniques, software applications/websites, (e.g., social and/or business networking websites, such as Facebook®, LinkedIn®, Google+®, Twitter®, etc., business applications, etc.), programming languages, etc., while ensuring compatibility with changing technologies, parameters, protocols, standards, etc.

It is contemplated that any number and type of components may be added to and/or removed from monitoring mechanism 110 and/or monitoring elements 112 to facilitate various embodiments including adding, removing, and/or enhancing certain features. For brevity, clarity, and ease of understanding of monitoring mechanism 110 and monitoring elements 112, many of the standard and/or known components, such as those of a computing device, are not shown or discussed here. It is contemplated that embodiments, as described herein, are not limited to any particular technology, topology, system, architecture, and/or standard and are dynamic enough to adopt and adapt to any future changes.

FIG. 3A illustrates a transaction sequence 300 for facilitating non-invasive blood hemoglobin monitoring using non-invasive hemoglobin monitoring device 100 of FIG. 1 according to one embodiment. Transaction sequence 300 may be performed by processing logic that may comprise hardware (e.g., circuitry, dedicated logic, programmable logic, etc.), software (such as instructions run on a processing device), or a combination thereof. In one embodiment, transaction sequence 300 may be performed by monitoring mechanism 110 and/or monitoring elements 112 of monitoring device 100 of FIG. 1. The processes of transaction sequence 300 are illustrated in linear sequences for brevity and clarity in presentation; however, it is contemplated that any number of them can be performed in parallel, asynchronously, or in different orders.

Embodiments provide for monitoring device 100 having monitoring mechanism 110 and monitoring elements 112 of FIG. 1 for monitoring of blood hemoglobin in persons without having to pierce the skin (e.g., finger) or having the need for drawing blood. Referring to various components of monitoring mechanism 110 and/or monitoring elements 112 of monitoring device 100 of FIG. 2, in one embodiment, method 300 begins at processing block 301 with a light source, such as photo/light source 237 of FIG. 2, transmitting light at a fixed wavelength through an emitting focused beam, at block 303, and further through a receiving focused beam where the emitted light is to be received at a photo/light sensor, such as light sensor 241 of FIG. 2, at block 311.

In one embodiment, at 305, a light path is generated and, at 307, when a finger is placed at a placement area, such as placement area 231 of FIG. 2, the finger interrupts the light path while the light passes through the finger as it travels or attempts to travel through the two focused beams, e.g., emitting and receiving beams, at block 309.

In one embodiment, at block 313, the light may be receive or recognized at a peripheral interface controller, such as peripheral interface controller 233 of FIG. 2, for further processing, such as where each interruption may be detected as an analog signal (and recorded as an observation reading) by detection (interruption) logic 201 of FIG. 2 at block 315. At block 317, calibration of analog signals may be performed (such as the analog signals may be converted into digital signals and raw values may be extracted from the digital signal and subsequently, one or more average raw values may be computed based on the raw values, etc.) via calibration logic 209 of FIG. 2. In one embodiment, additional calibration may be performed at block 319 where absolute values are computed via absolute value computation logic 211 of FIG. 2, where the absolute values are computed based on the raw values and further, an average absolute value may be obtained based on the absolute values.

In some embodiments, at block 321, using sample counter 215 of FIG. 2, the aforementioned processes (e.g., detection of interruptions and their corresponding values, conversion of signals, extraction of raw values, computation of average raw values, etc.) may be performed or repeated for any number of times as deem appropriate or necessary by a user (e.g., patient placing the finger, medical personnel, etc.). Any data (e.g., values, readings, samples, etc.) received from the multiple iteration of these processes may be averaged for a smoothing value. At block 323, using sampling device and presentation logic 217 of FIG. 2, this smoothing value, which is sampled through one or more iterations of the aforementioned processes, is than converted into a final hemoglobin reading from the average absolute value, where the final hemoglobin reading represents the amount or concentration or level of hemoglobin in the blood. This final hemoglobin reading may then be presented or display via a display device/screen, such as display screen 245 of FIG. 2.

FIG. 3B illustrates a transaction sequence 340 for facilitating non-invasive blood hemoglobin monitoring using non-invasive hemoglobin monitoring device 100 of FIG. 1 according to one embodiment. Transaction sequence 340 may be performed by processing logic that may comprise hardware (e.g., circuitry, dedicated logic, programmable logic, etc.), software (such as instructions run on a processing device), or a combination thereof. In one embodiment, transaction sequence 340 may be performed by monitoring mechanism 110 and/or monitoring elements 112 of monitoring device 100 of FIG. 1. The processes of transaction sequence 340 are illustrated in linear sequences for brevity and clarity in presentation; however, it is contemplated that any number of them can be performed in parallel, asynchronously, or in different orders.

Transaction sequence 340 begins at block 341 with an initiation of one or more software programs (e.g., initiating one or more components of monitoring mechanism 110 and/or monitoring elements 112) wherein the initiation may be triggered with turning-on of power by switching-on a power button of monitoring device 100 at block 341. As the power button is turned-on, at block 343, calibration of one or more hardware components (e.g., calibrating one or more components of monitoring mechanism 110 and/or monitoring elements 112) of monitoring device 100 is performed. In some embodiments, calibration may be triggered with a body part, such as a finger, is placed in a placement area, such as placement area 231 of FIG. 2.

As described previously, at block 345, detection of light absorbance through one or more photo transistors of a reception control component of a light sensor, such as reception control component 243 of light sensor 241 of FIG. 2. The transmitted analog signals may then be processed to be converted into digital signals, such as digital pulses, at block 347. Each cycle of digital pulses may be counted as an individual sample by sample counter such that sample counter counts a number of samples required for a single reading at block 349.

At block 351, the counter counts the number to sample and a determination is made as to whether the number of samples are less than or equal to a particular number, such as 10, etc. If yes, the process continues a block 347 with digital signal processing, but if not, the process moves forward at block 353 and continues with performing averaging of the samples to obtain a smoothing value. Stated differently, after the set number of cycles, such as 10 cycles, is performed, the sample counter may automatically stop taking samples or converting the analog signals into digital signals. This may be followed by initiating the mathematical algorithm in which a variable may be used for calculating an average value of the collected samples. The average value may then be used to obtain a final hemoglobin reading indicating the concentration of hemoglobin in the blood. At block 355, the final hemoglobin reading may then be displayed at a display device/screen, such as display screen 245 of FIG. 2, and subsequently, the process ends at block 357.

FIG. 3C illustrates a method 360 for facilitating non-invasive blood hemoglobin monitoring using non-invasive hemoglobin monitoring device 100 of FIG. 1 according to one embodiment. Method 360 may be performed by processing logic that may comprise hardware (e.g., circuitry, dedicated logic, programmable logic, etc.), software (such as instructions run on a processing device), or a combination thereof. In one embodiment, method 360 may be performed by monitoring mechanism 110 and/or monitoring elements 112 of monitoring device 100 of FIG. 1. The processes of method 360 are illustrated in linear sequences for brevity and clarity in presentation; however, it is contemplated that any number of them can be performed in parallel, asynchronously, or in different orders.

Embodiments provide for monitoring device 100 having monitoring mechanism 110 and monitoring elements 112 of FIG. 1 for monitoring of hemoglobin levels in blood without having to pierce the skin (e.g., finger) or having the need for drawing blood. Referring to various components of monitoring mechanism 110 and/or monitoring elements 112 of monitoring device 100 of FIG. 2, in one embodiment, method 360 begins at block 361 when turning on of a non-invasive hemoglobin monitoring device, such as monitoring device 100 of FIG. 1, by turning on an on-off switch. At block 363, upon turning on of the monitoring device, an infrared light is generated at a photo or light source (e.g., infrared LED) within the monitoring device and passes through, for example, a couple of light beams, such as an emitting bean and a receiving beam, before reaching a photo or light sensor also within the monitoring device. In one embodiment, the infrared light may be of different wavelength as deem necessary and appropriate based on one or more factors described earlier in this document; for example, these wavelengths may range from (but not limited to) 640 nm to 1000 nm and, for example, a particular wavelength, such as 940 nm, may be chosen from the range.

At block 365, a finger (such as an index finger or any other finger or a thumb, a toe, etc.) of a user (e.g., any individual, such a healthy individual, a patient, etc.) may be placed within a placement area of the monitoring device to facilitate hemoglobin monitoring of the user. In some embodiments, the placement area may contain one or more sensors, such as biometric sensor, to sense the human finger and other features relating to the user, such as fingerprints, etc., that can reveal certain information about the user, such as their age, gender, race, ethnicity, medical history, such as blood pressure, allergies, medicine history, cardiovascular problems, blood sugar/glucose level and/or its history, previous hemoglobin readings, etc. At block 367, in one embodiment, the interruption in the light flow is detected as the infrared light is interrupted by the finger being placed in the placement area which is in the path of the light flowing on the emitting and receiving beams.

At block 369, observation readings relating to any number of light interruptions are detected and read in a wave form, such as in the form of analog signals. For example, three samples of 5 interruptions may be observed and read within a period of 5 seconds, etc., such as sample 1=987, 997, 993, 995, 990, sample 2=990, 987, 993, 995, 990, and sample 3=992, 987, 993, 995, 990. At block 371, these analog signals are converted into digital signals, such as, continuing with the previous example, converting 3 samples of 5 analog signals into 3 samples of 5 digital signals. In one embodiment, at block 373, the values obtained from the digital signals are put through one or more processes using one or more of monitoring elements 112 and/or monitoring mechanism 110, including algorithms, software programs, and/or mathematical formulae, as discussed with reference to FIG. 2 so that the values may be averaged to produce a single raw value. At block 375, the processes relating to blocks 369 and 371 may be repeated multiple times (e.g., 3 times for the aforementioned 3 samples) to obtain multiple sets of signals and their corresponding values to produce multiple raw values (e.g., if the processes are repeated 3 times, then 3 raw values may be obtained), such as raw value 1=992.4, raw value 2=991, and raw value 3=991.4.

In one embodiment, at block 377, the raw values may then be put through another of processes using one or more of monitoring elements 112 and/or monitoring mechanism 110, including algorithms, software programs and/or mathematical formulae, as discussed with reference to FIG. 2 to generate corresponding absolute values and further, any errors associated with any of the absolute values are detected and rectified by applying different coefficients to the various processes or processing algorithms. At block 379, an average of the absolute values is obtained. For example, continuing with the previous example, 3 absolute values may be generated based on the 3 raw values and then a single average absolute value may be obtained from averaging the 3 absolute values. At block 381, in one embodiment, the average absolute value is then converted or computed into a final hemoglobin reading indicating the concentration of hemoglobin in the blood of the user placing the finger in the placement area.

In one embodiment, this average absolute value may then be applied to one or more formulate or algorithms to compute a final hemoglobin reading, where the formulae may take into consideration any number and type of values, such as (but not limited to) time periods, observations readings, raw values, absolute values, etc., and other values, variables, coefficients, and constants, etc., to arrive to the final glucose reading. For example, in one embodiment, multiple hemoglobin (“Hb”, “Hgb”, or “HB”) readings may be achieved, such as 12.63 g/dL may be reached by multiplying the aforementioned average 992.4 into 0.114 and the deducting from 100.5. Similarly, other hemoglobin readings may be calculated to then compute the final hemoglobin reading. For example, hemoglobin Hb 1=(0.114×992.4)−(100.5)=12.63 g/dL; Hb 2=(0.114×991)−(100.5)=12.47 g/dL; and Hb 3=(0.114×991.4)−(100.5)=12.52 g/dL. The final hemoglobin reading may then be calculated by obtaining an average of the previously-calculated multiple Hb values, such as Final Hb=(12.63+12.47+12.52)/3=12.54 g/dL. This final hemoglobin reading is then displayed at a display screen at block 383.

FIG. 4A illustrates a front/side view of monitoring device 100 of FIG. 1 according to one embodiment. It is to be noted that for the sake of brevity, clarity, and ease of understanding, several details already discussed with reference to the preceding FIGS. 1-3B are not discussed or repeated here with reference to FIGS. 4A-4D. In the illustrated embodiment, monitoring device 100 may include monitoring mechanism 110 and monitoring elements 112 of FIG. 1 to perform one or more tasks to facilitate non-invasive blood hemoglobin monitoring as described throughout this document, such as with reference to FIGS. 1-4. In one embodiment, monitoring device 100 may include a computing system having one or more processing devices, logic including and/or based on software, hardware, and/or any combination of software and hardware, such as firmware.

In the illustrated embodiment, a front/side view of monitoring device 100 is shown to have top chamber 401 and bottom chamber 403. As illustrated, a symmetrical portion from both top and bottom chambers 401, 403 may be removed to make place for placement area 231 where, for example, a finger may be placed for monitoring of hemoglobin. As aforementioned, embodiments provide for novel and innovative technique for monitoring of hemoglobin without having to follow the conventional techniques of piercing or pinching fingers with a needle like instrument to obtain one or more drops of blood for testing purposes. In one embodiment, top and bottom chambers 401, 403 may be connected or joined together in the back with a roller-like connector 405 so that the two chambers 401, 403 may be easily opened or closed for easy placement of fingers, thumbs, toes, etc.

For example, FIG. 4B illustrates a side view of monitoring device 100 of FIG. 1 having a finger 494 (e.g., a human finger) placed in placement area 231 while top and bottom chambers 401, 403 and brought together such that finger 494 is firmly, yet gently, held in place to interrupt the infrared light running on beams between top and chambers 401, 403 as further described with reference to FIG. 2. Once a number of observations reading have been taken or a given time period for testing has expired, top and bottom chambers 401, 403 may then be pulled away from each other to release finger 494 as illustrated in FIG. 4B. As aforementioned with respect to FIG. 2, placement area 231 may include one or more sensors, such as biometric sensor 247, etc.

FIG. 4C further illustrates a top/back view of monitoring device 100 of FIG. 1 showing display device/screen 245, as part of top chamber 401, to display readings relating to monitoring of glucose, hemoglobin, heart rate, body temperature, blood pressure, etc., as well as other information, such as patient name, identification number, age, medical history, historical final readings in numbers or text or graphs or charts, lights or symbols (e.g., circles, bars, animated figures, etc., for providing messages or warnings (e.g., red circle for a hemoglobin reading that is too high, yellow flashing light for a hemoglobin reading that is too low, a happy face for normal, etc.), and the like. Display screen 245 may further display other relevant information, such as real-time number of observation readings, monitoring time period in real-time, current time, current outside or room temperature, names or identification numbers of medical personnel (e.g., patient's doctor, nurse, etc.), and the like.

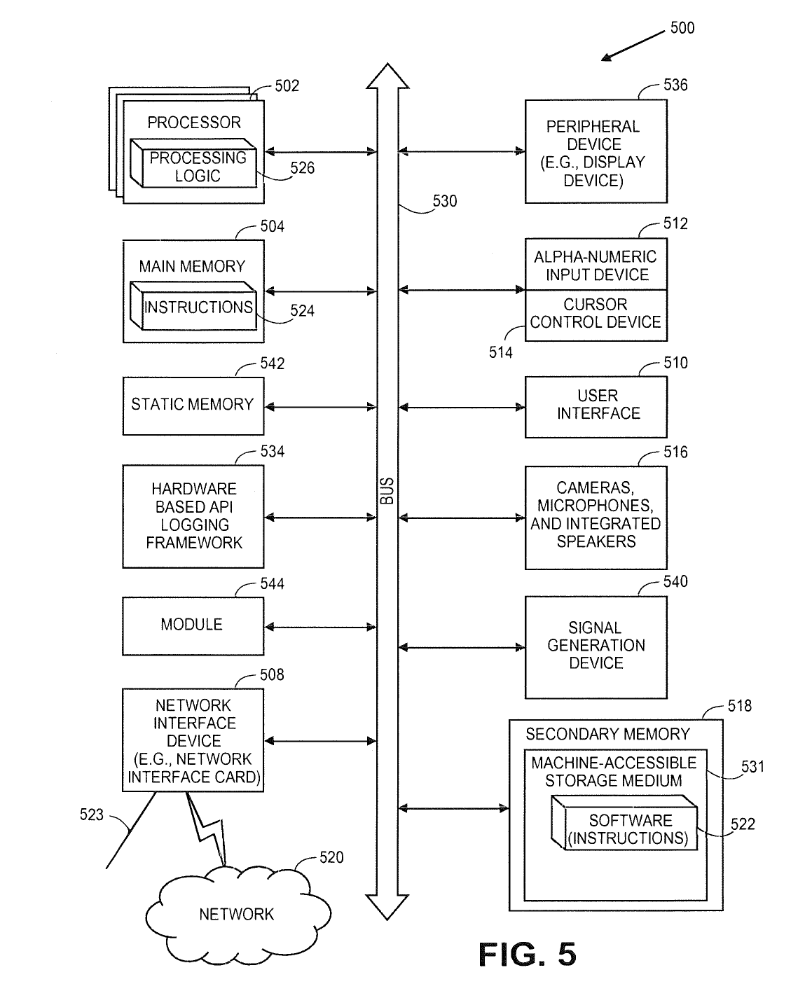

Now referring to FIG. 4D, it illustrates an unassembled view of monitoring device 100 of FIG. 1. As illustrated, monitoring device 100 includes top chamber 401, bottom chamber 403, connector 405, base 407, placement area 231 including top portion 411 that is attached to top chamber 401 and bottom portion 413 that is attached to bottom chamber 403, display device/screen 245, and processor 102 which may the same as or similar to processor 502 of FIG. 5. In one embodiment and as further described with reference to FIG. 2, monitoring elements 112 may be placed in any number of places within or coupled to monitoring device 100. For example, display screen 245 may be part of top chamber 401, as illustrated, or another part of monitoring device 100 or a separate display device (e.g., compute monitor, camera display, television, medical equipment screen, etc.) may be coupled to or placed in communication with monitoring device 100. Similarly, processor 102 may be part of top chamber 501, as illustrated, or bottom chamber 503 or, for example, a separate computing device may be coupled to or placed in communication with monitoring device 100.

Further, display screen 245 may also be used to serve as a user interface (e.g., GUI, WUI, touchscreen, etc.) for inputting and/or outputting information, such as user (e.g., patient) data including, for example, name, identification number, historical figures, names or codes of prescription drugs, date of last checkup, doctor/nurse name, etc. In one embodiment, display screen 245 may include a touchscreen (e.g., an interactive touchscreen) for inputting, outputting, editing, etc., information by touching display screen and further, display screen may offer a virtual keyboard that may be touched input information and set user preferences (e.g., font size, color, clock or no clock, overall user preference of data/information to be displayed via display screen 245, etc.).

It is contemplated that top and bottom chambers 401, 403 and various other parts of monitoring device 100 may be made from any number and type of materials, such as plastic, rubber, silicon, glass, iron, steel, etc., or any combination thereof and that monitoring device 100 is not limited to any particular number or type of material. It is contemplated that monitoring device 100 may further include other monitoring elements 112, such as peripheral interface controller 233 (e.g., inside bottom chamber 503), adjustment control component 233 (e.g., externally at top chamber 501), light source 237 including emission control component 239 (e.g., inside top chamber 501), light sensor 241 including reception control component 243 (e.g., inside bottom chamber 503), etc. Moreover, any number of components or parts (e.g., one or more of processors, memory, operating systems, display screens, sensors, cables, connectors, scanners, sensors, readers, etc.) may be added to or removed from monitoring device 100 to perform various tasks relating to non-invasive blood hemoglobin monitoring as described throughout this document.

FIG. 5 illustrates a diagrammatic representation of a machine 500 in the exemplary form of a computer system, in accordance with one embodiment, within which a set of instructions, for causing machine 500 to perform any one or more of the methodologies discussed herein, may be executed. Machine 500 may be the same as or similar to or contained within monitoring device 100 employing monitoring mechanism 110 and/or monitoring elements 112 of FIG. 1 according to one embodiment. In alternative embodiments, machine 100 may be connected (e.g., networked) to other machines either directly, such as via media slot or over a network, such as a cloud-based network, a Local Area Network (LAN), a Wide Area Network (WAN), a Metropolitan Area Network (MAN), a Personal Area Network (PAN), an intranet, an extranet, or the Internet. The machine may operate in the capacity of a server or a client machine in a client-server network environment, or as a peer machine in a peer-to-peer (or distributed) network environment or as a server or series of servers within an on-demand service environment, including an on-demand environment providing multi-tenant database storage services.

Certain embodiments of the machine may be in the form of a personal computer (PC), a tablet PC, a set-top box (STB), a Personal Digital Assistant (PDA), a cellular telephone, a web appliance, a server, a network router, switch or bridge, computing system, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein.

The exemplary computer system 500 includes one or more processors 502, a main memory 504 (e.g., read-only memory (ROM), flash memory, dynamic random access memory (DRAM) such as synchronous DRAM (SDRAM) or Rambus DRAM (RDRAM), etc., static memory 542, such as flash memory, static random access memory (SRAM), volatile but high-data rate RAM, etc.), and a secondary memory 518 (e.g., a persistent storage device including hard disk drives and persistent multi-tenant data base implementations), which communicate with each other via a bus 530. Main memory 504 includes instructions 524 (such as software 522 on which is stored one or more sets of instructions 524 embodying any one or more of the methodologies or functions of monitoring mechanism 110 and/or monitoring elements 112 of monitoring device 100 of FIG. 1 and other figures described herein) which operate in conjunction with processing logic 526 and processor 502 to perform the methodologies discussed herein.

Processor 502 represents one or more general-purpose processing devices such as a microprocessor, central processing unit, or the like. More particularly, the processor 502 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 502 may also be one or more special-purpose processing devices such as an application specific integrated circuit (ASIC), a field programmable gate array (FPGA), a digital signal processor (DSP), network processor, or the like. Processor 502 is configured to execute the processing logic 526 for performing the operations and functionality of monitoring mechanism 110 and/or monitoring elements 112 of monitoring device 100 of FIG. 1 and other figures discussed herein. Further, processor 502 and memory 504 may be the same as or similar to processor 102 and memory 104, respectively, of FIG. 1.

The computer system 500 may further include a network interface device 508, such as a network interface card (NIC). The computer system 500 also may include a user interface 510 (such as a video display unit, a liquid crystal display (LCD), or a cathode ray tube (CRT)), an alphanumeric input device 512 (e.g., a keyboard), a cursor control device 514 (e.g., a mouse), a signal generation device 540 (e.g., an integrated speaker), and other devices 516 like cameras, microphones, integrated speakers, etc. The computer system 500 may further include peripheral device 536 (e.g., wireless or wired communication devices, memory devices, storage devices, audio processing devices, video processing devices, display devices, etc.). The computer system 500 may further include a hardware-based application programming interface logging framework 534 capable of executing incoming requests for services and emitting execution data responsive to the fulfillment of such incoming requests.

Network interface device 508 may also include, for example, a wired network interface to communicate with remote devices via network cable 523, which may be, for example, an Ethernet cable, a coaxial cable, a fiber optic cable, a serial cable, a parallel cable, etc. Network interface device 508 may provide access to a LAN, for example, by conforming to IEEE 802.11b and/or IEEE 802.11g standards, and/or the wireless network interface may provide access to a personal area network, for example, by conforming to Bluetooth standards. Other wireless network interfaces and/or protocols, including previous and subsequent versions of the standards, may also be supported. In addition to, or instead of, communication via the wireless LAN standards, network interface device 508 may provide wireless communication using, for example, Time Division, Multiple Access (TDMA) protocols, Global Systems for Mobile Communications (GSM) protocols, Code Division, Multiple Access (CDMA) protocols, and/or any other type of wireless communications protocols.

The secondary memory 518 may include a machine-readable storage medium (or more specifically a machine-accessible storage medium) 531 on which is stored one or more sets of instructions (e.g., software 522) embodying any one or more of the methodologies or functions of monitoring mechanism 110 and/or monitoring elements 112 of FIG. 1 and other figures described herein. The software 522 may also reside, completely or at least partially, within the main memory 504, such as instructions 524, and/or within the processor 502 during execution thereof by the computer system 500, the main memory 504 and the processor 502 also constituting machine-readable storage media. The software 522 may further be transmitted or received over network 520 via the network interface card 508. The machine-readable storage medium 531 may include transitory or non-transitory machine-readable storage media.

Embodiments may be provided, for example, as a computer program product which may include one or more machine-readable or computer-readable media having stored thereon machine-executable or computer-executable instructions that, when executed by one or more machines such as a computer, one or more processing devices, a network of computers, or other electronic devices, may result in the one or more machines carrying out operations in accordance with embodiments described herein. A machine-readable medium may include, but is not limited to, floppy diskettes, optical disks, CD-ROMs (Compact Disc-Read Only Memories), and magneto-optical disks, ROMs, RAMs, EPROMs (Erasable Programmable Read Only Memories), EEPROMs (Electrically Erasable Programmable Read Only Memories), magnetic or optical cards, flash memory, or other type of media/machine-readable medium suitable for storing machine-executable instructions.

Moreover, embodiments may be downloaded as a computer program product, wherein the program may be transferred from a remote computer (e.g., a server) to a requesting computer (e.g., a client) by way of one or more data signals embodied in and/or modulated by a carrier wave or other propagation medium via a communication link (e.g., a modem and/or network connection).

Modules 544 relating to and/or include components and other features described herein (for example in relation to monitoring mechanism 110 and/or monitoring elements 112 of monitoring device 100 as described with reference to FIG. 1) can be implemented as discrete hardware components or integrated in the functionality of hardware components such as ASICS, FPGAs, DSPs or similar devices. In addition, modules 544 can be implemented as firmware or functional circuitry within hardware devices. Further, modules 544 can be implemented in any combination hardware devices and software components.

The techniques shown in the figures can be implemented using code and data stored and executed on one or more electronic devices (e.g., an end station, a network element). Such electronic devices store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals). In addition, such electronic devices typically include a set of one or more processors coupled to one or more other components, such as one or more storage devices (non-transitory machine-readable storage media), user input/output devices (e.g., a keyboard, a touchscreen, and/or a display), and network connections. The coupling of the set of processors and other components is typically through one or more busses and bridges (also termed as bus controllers). Thus, the storage device of a given electronic device typically stores code and/or data for execution on the set of one or more processors of that electronic device. Of course, one or more parts of an embodiment may be implemented using different combinations of software, firmware, and/or hardware.

References to “one embodiment”, “an embodiment”, “example embodiment”, “various embodiments”, etc., indicate that the embodiment(s) so described may include particular features, structures, or characteristics, but not every embodiment necessarily includes the particular features, structures, or characteristics. Further, some embodiments may have some, all, or none of the features described for other embodiments.

In the following description and claims, the term “coupled” along with its derivatives, may be used. “Coupled” is used to indicate that two or more elements co-operate or interact with each other, but they may or may not have intervening physical or electrical components between them.

As used in the claims, unless otherwise specified the use of the ordinal adjectives “first”, “second”, “third”, etc., to describe a common element, merely indicate that different instances of like elements are being referred to, and are not intended to imply that the elements so described must be in a given sequence, either temporally, spatially, in ranking, or in any other manner.

The following clauses and/or examples pertain to further embodiments or examples. Specifics in the examples may be used anywhere in one or more embodiments. The various features of the different embodiments or examples may be variously combined with some features included and others excluded to suit a variety of different applications. Examples may include subject matter such as a method, means for performing acts of the method, at least one machine-readable medium including instructions that, when performed by a machine cause the machine to performs acts of the method, or of an apparatus or system for facilitating hybrid communication according to embodiments and examples described herein.

Embodiment 1 that includes an apparatus to facilitate non-invasive and non-skin piercing monitoring of blood hemoglobin, comprising: a placement area to receive a body part including a finger, wherein the body part in the placement area causes one or more sets of interruptions in the running of a light, wherein each set of interruptions includes a plurality of interruptions; observation/reading logic to detect initial readings corresponding to interruptions of the one or more sets of interruptions, the initial readings including signals, wherein an analog signal is generated each time the light is interrupted while passing through the body part; absolute value computation module of the calibration logic to calculate absolute values based on average raw values, wherein the absolute value computation module is further to compute an average absolute value based on the absolute values; and predictive analysis logic to compute a final hemoglobin reading based on the average absolute value.

Embodiment 2 includes the subject matter of Embodiment 1, further comprising: a light source to emit the light within the apparatus, wherein the light is received at a light sensor and runs in beams including an emitting beam and a receiving beam; and raw value computation module of calibration logic to calculate raw values based on the initial readings, wherein the raw value computation module if further to compute the average raw values based on the raw values, wherein each of the average raw value corresponds to a set of the one or more sets of interruptions, wherein the final hemoglobin reading is computed without having to pierce or pinch the body part.

Embodiment 3 includes the subject matter of Embodiment 1, further comprising: sampling device and presentation logic to prepare the final hemoglobin reading for presentation at a display screen, wherein the display screen to display the hemoglobin reading.

Embodiment 4 includes the subject matter of Embodiment 1, further comprising: detection (interruption) logic to detect the interruptions of the one or more sets of interruptions causing the signals, wherein the signals include analog signals.

Embodiment 5 includes the subject matter of Embodiment 4, further comprising: signal conversion logic to convert the analog signals into digital signals, wherein the raw values are computed based on the initial readings including the digital signals.

Embodiment 6 includes the subject matter of Embodiment 1, further comprising: a sample counter to facilitate and maintain a count of the one or more sets of interruptions to generate one or more reading samples corresponding to the one or more sets of interruptions, wherein the sample counter is further to initiate or continue or terminate the count when the count is greater than or equal to or less than a predetermined value.

Embodiment 7 includes the subject matter of Embodiment 1, further comprising: error rectification module of the calibration logic to identify and rectify one or more errors associated with the computation of the absolute values.

Embodiment 8 includes the subject matter of Embodiment 1, wherein the light source includes an emission control component in a top chamber or a bottom chamber of the apparatus to emit the light, and wherein the light sensor includes a reception control component in the top chamber or the bottom chamber of the apparatus to receive the light.

Embodiment 9 that includes a method for facilitating non-invasive and non-skin piercing monitoring of blood hemoglobin comprising: receiving a body part including a finger, wherein the body part in the placement area causes one or more sets of interruptions in the running of a light, wherein each set of interruptions includes a plurality of interruptions; detecting initial readings corresponding to interruptions of the one or more sets of interruptions, the initial readings including signals, wherein an analog signal is generated each time the light is interrupted while passing through the body part; calculating absolute values based on average raw values, wherein calculating includes computing an average absolute value based on the absolute values; and computing a final hemoglobin reading based on the average absolute value.

Embodiment 10 includes the subject matter of Embodiment 9, further comprising: emitting the light within a hemoglobin monitoring device, wherein the light is received at a light sensor and runs in beams including an emitting beam and a receiving beam; and calculating raw values based on the initial readings, wherein calculating includes computing the average raw values based on the raw values, wherein each of the average raw value corresponds to a set of the one or more sets of interruptions, wherein the final hemoglobin reading is computed without having to pierce or pinch the body part.

Embodiment 11 includes the subject matter of Embodiment 9, further comprising: preparing the final hemoglobin reading for presentation at a display screen; and displaying, via the display screen, the final hemoglobin reading.

Embodiment 12 includes the subject matter of Embodiment 9, further comprising: detecting the interruptions of the one or more sets of interruptions causing the signals, wherein the signals include analog signals.

Embodiment 13 includes the subject matter of Embodiment 12, further comprising: converting the analog signals into digital signals, wherein the raw values are computed based on the initial readings including the digital signals.

Embodiment 14 includes the subject matter of Embodiment 9, further comprising: facilitating and maintaining a count of the one or more sets of interruptions to generate one or more reading samples corresponding to the one or more sets of interruptions; and initiating or continuing or terminating the count when the count is greater than or equal to or less than a predetermined value.

Embodiment 15 includes the subject matter of Embodiment 9, further comprising: identifying and rectifying one or more errors associated with the computation of the absolute values.

Embodiment 16 includes at least one machine-readable medium comprising a plurality of instructions, when executed on a computing device, to implement or perform a method or realize an apparatus as claimed in any preceding claims.

Embodiment 17 includes at least one non-transitory or tangible machine-readable medium comprising a plurality of instructions, when executed on a computing device, to implement or perform a method or realize an apparatus as claimed in any preceding claims.

Embodiment 18 includes a system comprising a mechanism to implement or perform a method or realize an apparatus as claimed in any preceding claims.

Embodiment 19 includes an apparatus comprising means to perform a method as claimed in any preceding claims.

Embodiment 20 includes a computing device arranged to implement or perform a method or realize an apparatus as claimed in any preceding claims.

Embodiment 21 includes a communications device arranged to implement or perform a method or realize an apparatus as claimed in any preceding claims.

Embodiment 22 includes a system comprising: receiving a body part including a finger, wherein the body part in the placement area causes one or more sets of interruptions in the running of a light, wherein each set of interruptions includes a plurality of interruptions; detecting initial readings corresponding to interruptions of the one or more sets of interruptions, the initial readings including signals, wherein an analog signal is generated each time the light is interrupted while passing through the body part; calculating absolute values based on average raw values, wherein calculating includes computing an average absolute value based on the absolute values; and computing a final hemoglobin reading based on the average absolute value.

Embodiment 23 includes the subject matter of Embodiment 22, further comprising: emitting the light within a hemoglobin monitoring device, wherein the light is received at a light sensor and runs in beams including an emitting beam and a receiving beam; and calculating raw values based on the initial readings, wherein calculating includes computing the average raw values based on the raw values, wherein each of the average raw value corresponds to a set of the one or more sets of interruptions, wherein the final hemoglobin reading is computed without having to pierce or pinch the body part.

Embodiment 24 includes the subject matter of Embodiment 22, further comprising: preparing the final hemoglobin reading for presentation at a display screen; and displaying, via the display screen, the final hemoglobin reading.

Embodiment 25 includes the subject matter of Embodiment 22, further comprising: detecting the interruptions of the one or more sets of interruptions causing the signals, wherein the signals include analog signals.

Embodiment 26 includes the subject matter of Embodiment 25, further comprising: converting the analog signals into digital signals, wherein the raw values are computed based on the initial readings including the digital signals.

Embodiment 27 includes the subject matter of Embodiment 22, further comprising: facilitating and maintaining a count of the one or more sets of interruptions to generate one or more reading samples corresponding to the one or more sets of interruptions; and initiating or continuing or terminating the count when the count is greater than or equal to or less than a predetermined value.

Embodiment 28 includes the subject matter of Embodiment 22, further comprising: identifying and rectifying one or more errors associated with the computation of the absolute values.

Embodiment 29 includes an apparatus comprising: means for receiving a body part including a finger, wherein the body part in the placement area causes one or more sets of interruptions in the running of a light, wherein each set of interruptions includes a plurality of interruptions; means for detecting initial readings corresponding to interruptions of the one or more sets of interruptions, the initial readings including signals, wherein an analog signal is generated each time the light is interrupted while passing through the body part; means for calculating absolute values based on average raw values, wherein calculating includes computing an average absolute value based on the absolute values; and means for computing a final hemoglobin reading based on the average absolute value.

Embodiment 30 includes the subject matter of Embodiment 29, further comprising: means for emitting the light within a hemoglobin monitoring device, wherein the light is received at a light sensor and runs in beams including an emitting beam and a receiving beam; and means for calculating raw values based on the initial readings, wherein calculating includes computing the average raw values based on the raw values, wherein each of the average raw value corresponds to a set of the one or more sets of interruptions, wherein the final hemoglobin reading is computed without having to pierce or pinch the body part.

Embodiment 31 includes the subject matter of Embodiment 29, further comprising: means for preparing the final hemoglobin reading for presentation at a display screen; and means for displaying, via the display screen, the final hemoglobin reading.

Embodiment 32 includes the subject matter of Embodiment 29, further comprising: means for detecting the interruptions of the one or more sets of interruptions causing the signals, wherein the signals include analog signals.

Embodiment 33 includes the subject matter of Embodiment 32, further comprising: means for converting the analog signals into digital signals, wherein the raw values are computed based on the initial readings including the digital signals.

Embodiment 34 includes the subject matter of Embodiment 29, further comprising: means for facilitating and maintaining a count of the one or more sets of interruptions to generate one or more reading samples corresponding to the one or more sets of interruptions; and means for initiating or continuing or terminating the count when the count is greater than or equal to or less than a predetermined value.

Embodiment 35 includes the subject matter of Embodiment 29, further comprising: means for identifying and rectifying one or more errors associated with the computation of the absolute values.

Embodiment 38 includes medical device including a non-invasive non-piercing blood hemoglobin monitoring device arranged to implement or perform a method or realize an apparatus as claimed in any preceding claims.

Any of the above embodiments may be used alone or together with one another in any combination. Embodiments encompassed within this specification may also include embodiments that are only partially mentioned or alluded to or are not mentioned or alluded to at all in this brief summary or in the abstract. Although various embodiments may have been motivated by various deficiencies with the prior art, which may be discussed or alluded to in one or more places in the specification, the embodiments do not necessarily address any of these deficiencies. In other words, different embodiments may address different deficiencies that may be discussed in the specification. Some embodiments may only partially address some deficiencies or just one deficiency that may be discussed in the specification, and some embodiments may not address any of these deficiencies.

While one or more implementations have been described by way of example and in terms of the specific embodiments, it is to be understood that one or more implementations are not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements. It is to be understood that the above description is intended to be illustrative, and not restrictive.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

What is claimed is:

1. A blood-hemoglobin monitoring device, comprising:
a single light source of a single wavelength in either a top chamber or a bottom chamber of the blood-hemoglobin monitoring device;
a light sensor in either the top chamber or the bottom chamber opposite the light source;
a light-insulated finger-placement area between the top chamber and the bottom chamber, wherein a finger in the finger-placement area becomes an absorbing medium of light passing between the light source and the light sensor;
a processor disposed in either the top chamber or the bottom chamber, wherein the processor includes an absolute value computation module configured to calculate absolute values based on readings of the absorbing medium by the light sensor; and
predictive analysis logic configured to compute a final blood hemoglobin reading based on an average absolute value from the absolute values.
